(19) United States
(12) Patent Application Publication
(10) Pub. No.: US 2019/0151603 A1
(43) Pub. Date: May 23, 2019

(54) METHOD OF PROVIDING REMOTE PSYCHOLOGICAL AID

(71) Applicant: Pavel Pavlovich Horoshutin, Irkutsk (RU)

(72) Inventor: Pavel Pavlovich Horoshutin, Irkutsk (RU)

(21) Appl. No.: 16/255,176

(22) Filed: Jan. 23, 2019

Related U.S. Application Data
(63) Continuation-in-part of application No. PCT/RU2018/000295, filed on May 7, 2018.
Application No. PCT/RU2018/000295 is a continuation-in-part of application No. PCT/RU2017/000008, filed on Jan. 11, 2017.

(30) Foreign Application Priority Data
Jul. 25, 2016 (RU) 2016130360
Jan. 24, 2018 (RU) 2018102742

(51) Int. Cl. A61M 21/02; A61B 5/00; A61B 5/16; G09B 5/06

(52) U.S. Cl. CPC A61M 21/02; A61B 5/0022; A61M 2021/0016; G09B 5/06; A61B 5/165

(57) ABSTRACT

Methods and item kits for providing remote psychological aid are described herein. A method of providing remote psychological aid may commence with performing psychodiagnostics of a mental state and mental dependencies of a patient. The method may further include selecting, based on psychodiagnostics, an item kit for the patient to undergo an autogenic training. The item kit may include a plurality of items, one or more tools for use of one or more of the plurality of items, and instructions for using the item kit. Each of the plurality of items may evoke pleasant impressions in the patient through sensory channels of the patient. The method may further include providing the item kit to the patient. The method may continue with conducting the autogenic training by the patient using one or more of the plurality of items of the item kit.

### CROSS-REFERENCE TO RELATED APPLICATIONS

This application is a Continuation-in-part of PCT application No. PCT/RU2017/000008, entitled “METHOD OF PROVIDING REMOTE PSYCHOLOGICAL AID”, filed on Jan. 11, 2017, which claims priority to Russian Application No. 201613036, entitled “METHOD OF PROVIDING REMOTE PSYCHOLOGICAL AID”, filed on Jul. 25, 2016, and a Continuation-In-Part of PCT application No. PCT/RU2018/000295, entitled “METHOD OF PROVIDING REMOTE PSYCHOLOGICAL AID”, filed on May 7, 2018, which claims priority to Russian Application No. 2018102742, entitled “METHOD OF PROVIDING REMOTE PSYCHOLOGICAL AID”, filed on Jan. 24, 2018. The aforementioned applications are incorporated herein by reference in their entireties for all purposes.

### TECHNICAL FIELD

The present disclosure relates generally to psychology, and specifically to methods and item kits for providing remote psychological aid to solve problems related to mental states and dependencies and problematic social behavior.

### BACKGROUND

It is a known fact that more than half of patients of psychologists experience stress before the first meeting with a specialist. It is also known that direct contact of a psychologist with a patient when providing psychological aid often leads to the emergence of new forms of mental dependencies. Furthermore, there is a problem of providing psychological services to patients who live in remote settlements and have no opportunity to receive qualified psychological aid.

A method of interactive treatment of addictive behavior described in patent No. RU2252038, published in 2005, A61M21/00, includes providing psychotherapeutic, suggestive impact on a patient, affecting the patient by sound, setting an attitude related to the formation of negative emotions by prompting the patient to focus on unpleasant grotesquely emphasized moments of abuse by projecting, on a screen of audiovisual devices, a video sequence of negative pictures, and forming positive emotions in the patient when showing positive pictures. The psychotherapeutic, suggestive impact is provided through channels of audiovisual devices under constant remote control of psycho-emotional state of the patient and the level of addictive behavior attitude of the patient by the doctor. Firstly, initial psycho-physiological characteristics, fluctuations of the omega-potential, anxiety level, emotional reactions to relevant questions at rest and in presence of negative emotions are registered. Secondly, a dominance for a healthy lifestyle and a program for abandoning the dependencies is formed in the patient in the presence of positive emotions. The psycho-emotional state controlled according to the stability of psycho-physiological reactions is improved and the anxiety is reduced. Behavioral stereotypes are corrected with development of an adaptive self-realization strategy. Then, the patient by himself selects a motivating video image as a permanent screen saver of an audiovisual device to activate the previously formed dominance and the program for abandoning the dependencies. The verbal impact is performed in a stereophonic mode with binaural sounds vibrations in the vocal range and an adjustable frequency difference for the right and the left channels from 30 to 3 Hz.

A method described in patent No. RU2319517, published in 2008, A61M21/00, includes registration of psycho-physiological reactions of a patient, selection of a remote individual program for interactive correction of addictive behavior of the patient in the presence of persons interested in quitting addictive behavior by the patient, and selection of audio and video information including the attitude related to the eliminating the subject of dependency. The interested parties are preliminarily trained to assess a condition of the patient and perform a plan of action in case of a withdrawal behavior of the patient, upon which, in case of the withdrawal behavior, the interested persons use a mobile phone to dial a psychological service operator. At the beginning of treatment, transferring audio information of the interactive correction program is initiated, followed by turning on the video information of the interactive correction program, and then, upon change of the level of addictive behavior of the patient, correction of the interactive program is performed at the request of the patient himself or interested persons.

However, in the known methods, the psychodiagnostics takes place using a direct contact of a doctor with a patient and, therefore, the situation for the primary stress occurrence still exists. Even though the treatment process is further carried out remotely, it includes constant monitoring with a direct contact with an attending physician, which may cause the emergence of new mental dependencies. Furthermore, the known methods provide no possibility for applying the anonymity principle to treatment of the patient.

Based on the characteristics and analysis of the known methods, it is clear that the problem of creating less complex methods of providing remote psychological aid is currently important.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Provided are methods and item kits for providing remote psychological aid. In some example embodiments, a method of providing remote psychological aid may commence with performing psychodiagnostics of a mental state and mental dependencies of a patient to provide psychodiagnostics data associated with the patient. The method may further include selecting, based on the psychodiagnostics data, an item kit for the patient to undergo an autogenic training. The item kit may include a plurality of items, one or more tools for use of one or more of the plurality of items, and instructions on use of the item kit. Each of the plurality of items may evoke pleasant impressions in the patient through sensory channels of the patient. The method may further include providing the item kit to the patient. The method may continue with performing, upon receipt of the item kit by the patient, psychological correction of the mental state and the mental dependencies of the patient. The performance of the psychological correction may include conducting the autogenic training by the patient using one or more of the plurality of items of the item kit.

In some example embodiments, an item kit for providing remote psychological aid may include a plurality of items, one or more tools for use of one or more of the plurality of items, and instructions on use of the item kit. The plurality of items may include one or more of a text of an autogenic training for audio listening, at least one food additive, at least one aromatic essential oil, at least one mineral, and one or more images with a suggestive formula for changing an attitude to change a mental state and mental dependencies of a patient. The plurality of items may be selected based on psychodiagnostics data of the patient. The psychodiagnostics data may be obtained based on psychodiagnostics of the mental state and the mental dependencies of the patient. Each of the plurality of items may evoke pleasant impressions in the patient through sensory channels of the patient.

Additional objects, advantages, and novel features will be set forth in part in the detailed description section of this disclosure, which follows, and in part will become apparent to those skilled in the art upon examination of this specification and the accompanying drawings or may be learned by production or operation of the example embodiments. The objects and advantages of the concepts may be realized and attained by means of the methodologies, instrumentalities, and combinations particularly pointed out in the appended claims.

### DETAILED DESCRIPTION

The following detailed description includes references to the accompanying drawings, which form a part of the detailed description. The drawings show illustrations in accordance with exemplary embodiments. These exemplary embodiments, which are also referred to herein as “examples,” are described in enough detail to enable those skilled in the art to practice the present subject matter. The embodiments can be combined, other embodiments can be utilized, or structural, logical, and electrical changes can be made without departing from the scope of what is claimed. The following detailed description is, therefore, not to be taken in a limiting sense, and the scope is defined by the appended claims and their equivalents.

The present disclosure provides methods and item kits for providing remote psychological aid. A method of providing remote psychological aid is aimed at providing qualified psychological aid in the absence of direct contact of a patient with a specialist, such as a psychologist, by performing psychodiagnostics and psychological correction anonymously for the patient to correct mental states and mental dependencies of the patient. The method is directed to simplifying the process of provision of psychological aid to the patient having mental states and mental dependencies. The method is provided to work with patients who need qualified psychological aid, but live in remote settlements, and with patients who want to receive psychological aid remotely, or even anonymously.

The method of providing remote psychological aid may include performing psychodiagnostics of a mental state and mental dependencies of a patient to resolve problems of behavior related to the mental states and mental dependencies. Specifically, a request for the remote psychological aid may be received by a psychological center from the patient. The request may be received via long-distance communication means. In an example embodiment, the communication means may include one or more of the following: a mail, a phone, a delivery service, the Internet, and so forth.

Based on the request received from the patient, one or more tests to perform the psychodiagnostics may be sent to the patient via the communication means. Upon receipt of the one or more tests, the patient may perform the psychodiagnostics of the mental states and the mental dependencies by himself using the one or more tests. In other words, the testing of the patient may be performed by the patient himself without contacting a psychologist of the psychological center. In an example embodiment, the one or more tests may be directed to testing the patient to ascertain sensory preferences of the patient through sensory channels of a sensory system of the patient. Upon passing the one or more tests, the patient may send results of the one or more tests to the psychological center via the communication means.

The results of the one or more tests may serve as psychodiagnostics data of the patient. Based on the psychodiagnostics data, an item kit for the patient to undergo an autogenic training may be selected individually for the patient. The item kit may include a plurality of items acting as sensory stimuli for the sensory channels of the sensory system of the patient, one or more tools for use of one or more of the plurality of items, and instructions on use of the item kit. Each of the plurality of items may evoke pleasant impressions in the patient through the sensory channels of the patient. The selected item kit may be provided to the patient. Specially, the selected item kit may be sent to the patient by a delivery service or by mail.

Upon receipt of the item kit by the patient, psychological correction of the mental state and the mental dependencies of the patient may be performed. Specifically, the psychological correction may include conducting the autogenic training by the patient himself using one or more of the plurality of items of the item kit. Therefore, the psychodiagnostics and the psychological correction of mental states and mental dependencies of the patient may be performed without contacting the psychologist. Furthermore, the psychodiagnostics of the mental state of the patient may be performed anonymously. In an example embodiment, in accordance with the instructions on use, the patient may perform the autogenic training twice a day, in the morning and in the evening, for at least 10 minutes for at least 1 week.

The method of providing remote psychological aid may be performed as follows. A patient may register with a psychological center using a long-distance communication means (for example, a phone, Internet, mail, etc.). The psychological center may provide psychological aid to patients remotely, i.e., without a direct contact of patients with phycologists. In an example embodiment, the patient may register anonymously, i.e. without providing personal data. In some embodiments, the patient may send a request for psychological aid to a website of the psychological center.

After that, the patient may be remotely exposed to psychodiagnostics using tests. The psychological center may provide tests to the patient. The patient may answer the test questions by himself and send the results of the tests to the psychological center. After receiving the results of the tests, the psychologist of the psychological center may consider the results of the tests as psychodiagnostics data of the patient. The psychologist may use the psychodiagnostics data of the patient to develop a program of an autogenic training course individually for the patient.

The program of the autogenic training course may be developed by selecting an item kit for the patient to undergo an autogenic training. Specifically, to select items for the item kit, types of the items evoking pleasant impressions in the patient through the sensory channels may be determined. Furthermore, a number of the items required to maximally evoke the pleasant impressions in the patient through the sensory channels may be determined. The items of the item kit may include text of the autogenic training for audio listening, at least one food additive, at least one aromatic essential oil, at least one mineral, and one or more images with a suggestive formula for changing an attitude to change the mental state and the mental dependencies. In psychology, an attitude includes a set of emotions, behaviors, and beliefs towards a particular person, thing, object, or event.

The text of the autogenic training may be drafted individually for the patient based on the psychodiagnostics data of the patient and may be directed to changing the attitude, which may be a reason for the mental states and the mental dependencies of the patient. The selected item kit may further include tools, or accessories, for using one or more of the plurality of items, such as an aroma lamp for the aromatic essential oil and a voice recorder for recording the text by the patient.

The selected item kit and instructions for the use of items may be sent to the patient, for example, by a delivery service. Upon receipt of the item kit, the patient may review the instructions on use and perform necessary actions. In an example embodiment, the patient may record the text of the autogenic training, e.g., via an audio player, using a voice of the patient.

The patient may undergo the course of psychological correction by himself by applying the item kit based on the instructions. In case the patient has difficulties understanding the instructions, the patient may, for example, contact the psychological center to request clarifications with regard to the instructions on use of the item kit using the communication means, for example, by phone, or by mail. Additionally, the patient may request additional recommendations related to the autogenic training. In response to the requests of the patient, the clarifications with regard to the instructions on use or additional recommendations related to the autogenic training may be provided to the patient.

Sensory stimuli and a method of their selection are known, for example, from patents No. RU2315633 and No. RU2178204. A sample item kit having sensor stimuli is described, for example, in patent No. RU2632772.

In an example embodiment, the selection of an item kit based on the results of individual psychodiagnostics for the autogenic training course can be carried out as follows.

In the course of psychological correction of psychological states and dependencies, the psychologist may remotely test the patient to determine individual characteristics of the information perception sensory system of the patient to ascertain sensory preferences of the patient. The sensory preferences may include items evoking pleasant impressions through information perception sensory channels of the patient. Based on the testing of the patient, a number of sensory stimuli and their appearance that maximally evoke pleasant impressions may be determined.

In an example embodiment, the item kit selected for the patient may include the following items. Firstly, the item kit may include a set of essential oils and a device for using the essential oils, such as an aroma lamp. Particular essential oils may be selected by the patient in the course of remote psychodiagnostics and may be later used by the patient to create a persistent aroma in the room while undergoing the autogenic training by the patient. The essential oils may create a pleasant impression through an olfactory sensory channel of information perception.

Secondly, the item kit may include text of autogenic training. The text may be recorded by the patient himself using the audio player and then may be listened to by the patient, for example, daily. The text of the autogenic training may be drafted by the psychologist to change the attitude of the patient and create a pleasant impression in the patient through the auditory sensory channel of information perception.

Thirdly, the item kit may include one or more images with a suggestive formula. The suggestive formula may be selected to change the attitude of the patient. The first image may be an image made according to a design determined based on the psychodiagnostics. The first image may include one or more linguistic phrases of the suggestive formula to form a new attitude. The patient may choose a particular image, as well as color and text font, which are the most pleasant to him, by himself during the psychodiagnostics. The image may be selected to evoke the pleasant impressions in the patient through a visual sensory channel of information perception.

A second image may include a suggestive formula of kinesthetic sensations. The second image may be made according to a design determined based on the psychodiagnostics. The second image may include one or more linguistic phrases targeting kinesthetic sensations, the character of which may be also determined during the testing. The patient chooses a particular image, as well as color and text font, which are the most pleasant to him, by himself during the psychodiagnostics. The second image may evoke pleasant impressions through the kinesthetic sensory channel of information perception.

A third image may have a suggestive formula of reasoned persuasions. The third image may be made according to a design determined based on the psychodiagnostics. The patient selects the design and particular image from variants provided in tests by himself during the psychodiagnostics. The third image may be based on linguistic phrases, or reasoned persuasions. The reasoned persuasions may be also formed based on tests and may focus the attention of the patient during the autogenic training on pleasant reasoned persuasions. The third image may evoke the pleasant impressions through a reasoning sensory channel of information perception.

Fourthly, the item kit may include a food additive having a strong taste. The food additive may be a food additive pleasant for the patient, for example, lollipops, which may cause a long pleasant taste effect. The food additive may be selected during the psychodiagnostics by the patient from variants provided in tests. The food additive may be used by the patient while listening to the text of the autogenic training. The food additive may evoke the pleasant impressions in the patient through a gustatory sensory channel of the sensory system.

Fifthly, the item kit may include minerals of various shapes. The minerals may provide various tactile sensations. The minerals may be selected by the patient from variants provided in tests during the psychodiagnostics. The minerals may be held by the patient in his hand while listening to the text of the autogenic training. The minerals may evoke the pleasant impressions in the patient through a tactile sensory channel of the sensory system.

Sixthly, the item kit may include instructions on use of items. The instructions may include text explaining the order of application of the items of the item kit during an individual autogenic training of the patient.

The method of providing remote psychological aid may include the following steps. The method may commence with creating long-distance contactless communication channels between the patient and the psychologist, e.g., via mail, phone, or Internet. The method may further include performing psychodiagnostics of the mental state and mental dependencies of the patient using tests. Based on psychodiagnostics data, a program for conducting the autogenic training by the patient may be created. According to the program, an individual text of an autogenic training directed to changing the attitude may be drafted for the patient. The attitude may be the reason of undesirable mental dependencies, mental states, or social behavior. The method may further include an item kit for conducting the autogenic training by the patient. The method may continue with sending the item kit to a location of the patient. The method may further include administering the repeated listening to the audio of the text of the autogenic training by the patient with simultaneous applying of the items intended for conducting the autogenic training, such as holding a mineral in a hand by the patient, smelling aromatic essential oil dispersed in a room, eating a food additive, and looking at an image with a suggestive formula. In an example embodiment, the patient may perform the autogenic training every morning and every evening for at least 10 minutes for at least one week. The method may be directed to enhancing, on the subconscious level, the attitude relating to quitting addictive behavior that may be the reason for undesirable dependencies or mental states. The method may further be aimed at providing psychological rehabilitation to the patient having mental dependencies and mental states and correcting social behavior of the patient.

In practice, the method of providing remote psychological aid may be performed as follows. Firstly, a long-distance communication channel between a patient who applied to a psychological center for help and a psychologist may be created using, for example, a phone, mail, or the Internet. The method may further include performing psychodiagnostics of the mental state of the patient to determine the causes of the problematic behavior of the patient. The problematic behavior may be related, for example, to alcohol addiction. The method may continue with determining, based on the psychodiagnostics data, a list of positive impacts (sound, smell, images, etc.) on the patient. Then, a program for conducting an autogenic training by the patient may be developed. The program may include a sequence of particular actions prescribed for performance by the patient to provide psychological correction of mental states and mental dependencies of the patient. While developing the program, an individual text of an autogenic training directed to quitting problematic behavior by the patient may be drafted individually for the patient based on the psychodiagnostics data. The development of program may further include selecting an item kit for conducting the autogenic training by the patient, namely, selecting items, i.e. stimuli, for pleasant classical conditioning through all sensory channels of perception, selecting tools for using the items, and creating instructions for use of the items and the tools. The term ‘classical conditioning’ refers to a learning procedure in which a biologically potent stimulus (e.g., food) is paired with a previously neutral stimulus (e.g., a bell).

The method may continue with sending the item kit to a location of the patient. The method may further include providing repeated listening to the audio of the text of the autogenic training by the patient with the simultaneous use of items intended for conducting the autogenic training, for example, for two months every morning and every evening for 10 minutes.

The implementation of the method of remote psychological aid is illustrated by the following examples.

### Example 1. Psychological Correction of Dependencies

A 35-year-old man having a nicotine addiction and a desire to give up smoking applied for help. The man registered with the Internet site of the psychological center. After that, the man got remote access to a psychodiagnostics department and answered questions in 7 tests. Based on the answers of the man, information on the state of a sensory system of perception of the man was obtained, the attitude forming the basis for problematic behavior was determined, and sensory preferences of the man were identified. The received information was used to develop an autogenic training course with full-sensory classical conditioning aimed at formation of the behavior free from nicotine addiction. The package named “An item kit for full-sensory psychological rehabilitation” included: instructions for conducting the autogenic training, the text for self-suggestion, five sensory stimuli for pleasant classic conditioning, and tools for their use. The sensor stimuli included a flavored food additive, namely lollipops with mint flavor (for the gustatory sensory channel), one fir essential oil and an aromatic medallion for its use (for the olfactory sensory channel), jade beads (for the tactile sensory channel), a voice recorder and text of the autogenic training (for the auditory sensory channel), a table having a particular design with a suggestive formula (for the visual sensory channel, the reasoning sensory channel, and/or the kinesthetic sensory channel). The items were selected by the patient from several proposed options during testing as items evoking the most pleasant impressions in the patient.

The package was sent to the patient by an express delivery service. The patient called once to get clarification on the order of using the items. No further contacts with the patient occurred. No complaints or negative reviews have been received on the site.

### Example 2. Psychological Correction of States

A 29-year-old woman applied for help. The reason for applying for help was the aggressive behavior in the family and conflicts with the husband. Upon registering with the website and performing the remote psychodiagnostics, a map of a mental state of the woman was drawn up, causes of fear underlying the aggressive behavior were determined, and a psychological correction course was developed. Based on this, an item kit for remote psychological aid was formed and sent to the patient. Several calls were further received from the patient in which the patient asked for additional recommendations related to the autogenic training course. After three months, the patient left a positive review on the site.

### Example 3. Psychological Correction of Social Behavior

Upon publication of an announcement on provision of psychological services in a regional newspaper, a letter was received from a 58-year-old woman who requested psychological aid for her grandson, who did not want to go to school. Tests for psychodiagnostics were sent by letter to the address of the woman, for her grandson. After results of the tests passed by the grandson were received by the psychologist, a psychological correction program aimed at formation of a new type of behavior with a passion for learning was developed. A package with an item kit for full-sensory psychological rehabilitation was sent to the woman. The grandson took the autogenic training course under the control of his grandmother. The woman reported that the grandson stopped skipping classes at school.

### Example 4

A woman, 44 years old, having the problem of drug dependence applied for help. The attending physician who saw the evidence of this dependence in behavior of the woman suggested solving the problem of drug dependence with the help of psychologists. After registering with the website and undergoing a testing procedure by the woman, two dominant sensory channels of perception have been revealed, as well as a low level of behavioral attitudes associated with concerns about her health has been determined. Based on the psychodiagnostics data, an autogenic training course with two items of classical conditioning on two sensory perception channels and lasting for 6 minutes was proposed for performance by the woman daily, three times a day. The autogenic training course lasted for less than a week. Four days later, the woman communicated by phone with a specialist of the physiological center and informed about a significant improvement in her state. The woman never applied for further services again.

### Example 5

A mother of a young man, 22 years old, applied for help. The mother asked to solve the problem of gambling addiction. After performing all the previously mentioned procedures and testing, the dominance of sensory channels of the man was determined and classical items for each sensory channel were selected. An autogenic training course was developed for performance 4 times a day for 3 minutes. The autogenic training course lasted for more than a week. The problems with the motivation of the young man have occurred because the psychological correction procedure was imposed on the young man by his mother. The package with the item kit was sent to the mother with a request to follow the timely completion of the autogenic training course. Eight days later, the mother called and informed that the previous day the young man voluntarily refused, for the first time, to go to the gaming club.

### Example 6

A young family asked to solve the problems of harmonious relations in the family. In the process of testing, it is revealed that the problem of disharmony is the marital infidelity of the husband. Secondary testing of the husband was carried out, the sensory structure of information perception of the husband was determined, and items for classical conditioning were selected. An autogenic training course for the correction of marital infidelity was created. A package with the autogenic training course was sent to the family. Sessions of the autogenic training course lasted 3 times a day for 9 minutes. The autogenic training course lasted for less than a week. Five days after the autogenic training course has started, the wife called and noted with satisfaction that the husband was engaged in the autogenic training course with pleasure and that his attitude towards the family had noticeably improved.

### Example 7

A family, a husband, 34 years old, and a wife, 28 years old, applied for help. The conflict relationships in the family was associated with financial expenses. In the process of psychodiagnostics, it has been determined that the wife had a shopaholism dependency. The dominance of sensory channels of information perception of the woman was determined. Seven pleasant stimuli were selected for each sensory channel. A text of the autogenic training was written. A package with the autogenic training course including items for classical conditioning was sent to the patient. Training sessions were performed 1 time a day in the morning for 20 minutes. The autogenic training course lasted for 6 weeks. During the psychological correction, the patient never applied for consultations to the psychological center. After 6 weeks, a specialist from the psychological center called the patient and received confirmation that the autogenic training cause was effective for the psychological correction of the patient.

The method of providing remote psychological aid provides preparing an individual autogenic training course for a patient and implementing of the autogenic training course by the patient himself without a psychologist, thus enabling creating a safer, more convenient and less technically complex method of psychological correction of mental states and dependencies and correction of social behavior of the patient.

The introduction of a contactless and anonymous way into the existing practice of psychological counseling may help to provide professional psychological aid to a category of people who currently avoid contacts with psychologists and psychotherapists due to the stress and provide services to patients living in remote settlements and having no opportunity to receive psychological aid.

Thus, methods and item kits for providing remote psychological aid are described. Although embodiments have been described with reference to specific exemplary embodiments, it will be evident that various modifications and changes can be made to these exemplary embodiments without departing from the broader spirit and scope of the present application. Accordingly, the specification and drawings are to be regarded in an illustrative rather than a restrictive sense.

What is claimed is:

1. A method of providing remote psychological aid, the method comprising:
performing psychodiagnostics of a mental state and mental dependencies of a patient to provide psychodiagnostics data associated with the patient;
based on the psychodiagnostics data, selecting an item kit for the patient to undergo an autogenic training, wherein the item kit includes a plurality of items, one or more tools for use of one or more of the plurality of items, and instructions on use of the item kit, wherein each of the plurality of items evoke pleasant impressions in the patient through sensory channels of the patient;
providing the item kit to the patient; and
upon receipt of the item kit by the patient, performing psychological correction of the mental state and the mental dependencies of the patient, the performing the psychological correction including conducting the autogenic training by the patient using one or more of the plurality of items of the item kit.

2. The method of claim 1, further comprising:
receiving, via a communication means, a request for the remote psychological aid from the patient; and
based on the request, sending, via the communication means, one or more tests to perform the psychodiagnostics to the patient.

3. The method of claim 2, wherein the communication means includes one or more of the following: a mail, a phone, a delivery service, and the Internet.

4. The method of claim 2, wherein the request for the remote psychological aid is received from the patient using a website associated with a psychological center, the psychological center providing the remote psychological aid.

5. The method of claim 2, wherein the psychodiagnostics of the mental states and the mental dependencies is performed by the patient using the one or more tests without contacting a psychologist.

6. The method of claim 5, further comprising receiving, via the communication means, results of the one or more tests from the patient, wherein the psychodiagnostics data include the results of the one or more tests.

7. The method of claim 2, wherein the one or more tests include testing the patient to ascertain sensory preferences of the patient through the sensory channels of a sensory system of the patient.

8. The method of claim 7, wherein the selecting the item kit includes:
determining types of the items evoking pleasant impressions in the patient through the sensory channels; and
determining a number of the items required to maximally evoke the pleasant impressions in the patient through the sensory channels.

9. The method of claim 1, wherein the plurality of items includes one or more of the following:
a text of the autogenic training for audio listening;
at least one food additive;
at least one aromatic essential oil;
at least one mineral; and
one or more images with a suggestive formula for changing an attitude to change the mental state and the mental dependencies.

10. The method of claim 9, wherein the one or more tools for use of the one or more of the plurality of items include at least an aroma lamp for the at least one aromatic essential oil.

11. The method of claim 9, wherein the text of the autogenic training is recoded by the patient using a voice of the patient.

12. The method of claim 9, wherein the selecting the item kit includes drafting, based on the psychodiagnostics data, the text of the autogenic training for audio listening to change the attitude, the attitude being a reason for the mental states and the mental dependencies.

13. The method of claim 1, wherein the providing the item kit to the patient includes sending the item kit to the patient via a communication means.

14. The method of claim 1, wherein the psychodiagnostics is performed anonymously.

15. The method of claim 1, further comprising:
receiving one or more further requests from the user, the one or more further requests being associated with one or more of receiving clarifications with regard to the instructions on use of the item kit and receiving recommendations related to the autogenic training; and
in response to the one or more further requests, providing one or more of the clarifications with regard to the instructions on use and the recommendations related to the autogenic training of the patient.

16. An item kit for providing remote psychological aid, the item kit comprising:
a plurality of items, the plurality of items including one or more of:
a text of an autogenic training for audio listening;
at least one food additive;
at least one aromatic essential oil;
at least one mineral; and
one or more images with a suggestive formula for changing an attitude to change a mental state and mental dependencies of a patient;
one or more tools for use of one or more of the plurality of items; and
instructions on use of the item kit;
wherein the plurality of items are selected based on psychodiagnostics data of the patient, the psychodiagnostics data being obtained based on psychodiagnostics of the mental state and the mental dependencies of the patient;
wherein each of the plurality of items evoke pleasant impressions in the patient through sensory channels of the patient.

17. The item kit of claim 16, wherein the at least one food additive is selected to evoke the pleasant impressions in the patient through a gustatory sensory channel of the sensory system, the at least one aromatic essential oil is selected to evoke the pleasant impressions in the patient through an olfactory sensory channel of the sensory system, the at least one mineral is selected to evoke the pleasant impressions in the patient through a tactile sensory channel of the sensory system, the text of the autogenic training is drafted to evoke the pleasant impressions in the patient through an auditory sensory channel of the sensory system, and the one or more images are selected to evoke the pleasant impressions in the patient through one or more of a visual sensory channel, a reasoning sensory channel, and a kinesthetic sensory channel of the sensory system.

18. The item kit of claim 17, wherein the one or more images include at least one of the following images:
a first image, wherein the suggestive formula of the first image includes one or more linguistic phrases forming the attitude, wherein the first image evokes the pleasant impressions through a visual sensory channel of information perception of the patient;
a second image, wherein the suggestive formula of the second image includes one or more linguistic phrases targeted at kinesthetic sensations, and wherein the second image evokes the pleasant impressions through a kinesthetic sensory channel of information perception of the patient; and
a third image, wherein the suggestive formula of the second image includes one or more reasoned persuasions, and wherein the third image evokes the pleasant impressions through a reasoning sensory channel of information perception of the patient.

19. The item kit of claim 16, wherein the text of the autogenic training is drafted for the patient based on the psychodiagnostics data intended to change the attitude, the attitude being a reason for the mental states and the mental dependencies.

20. A method of providing remote psychological aid, the method comprising:
receiving, via a communication means, a request for the remote psychological aid from the patient;
based on the request, sending, via the communication means, one or more tests to perform the psychodiagnostics to the patient;
performing psychodiagnostics of a mental state and mental dependencies of a patient to provide psychodiagnostics data associated with the patient, wherein the psychodiagnostics of the mental states and the mental dependencies is performed by the patient using the one or more tests without contacting a psychologist;
further comprising receiving, via the communication means, results of the one or more tests from the patient, wherein the psychodiagnostics data include the results of the one or more tests;
based on the psychodiagnostics data, selecting an item kit for the patient to undergo an autogenic training, wherein the item kit includes a plurality of items, one or more tools for use of one or more of the plurality of items, and instructions for using the item kit, wherein each of the plurality of items evoke pleasant impressions in the patient through sensory channels of the patient, wherein the plurality of items includes one or more of the following: a text of the autogenic training for audio listening, at least one food additive, at least one aromatic essential oil, at least one mineral, and one or more images with a suggestive formula for changing an attitude to change the mental state and the mental dependencies;
providing the item kit to the patient; and
upon receipt of the item kit by the patient, performing psychological correction of the mental state and the mental dependencies of the patient, the performing the psychological correction includes conducting the autogenic training by the patient using one or more of the plurality of items of the item kit.
